(19) United States
(12) Patent Application Publication
(10) Pub. No.: US 2013/0011496 A1
(43) Pub. Date: Jan. 10, 2013

(54) CANCER TESTIS ANTIGENS AS BIOMARKERS IN NON-SMALL CELL LUNG CANCER

(75) Inventors: Jonathan S. Cebon, Clifton Hill (AU); Arun Azad, Melbourne (AU); Sid Deb, Melbourne (AU)

(73) Assignee: Ludwig Institute for Cancer Research Ltd., New York, NY (US)

(21) Appl. No.: 13/511,766

(22) PCT Filed: Nov. 23, 2010
(86) PCT No.: PCT/US10/57764

§ 371 Date: Sep. 18, 2012

Related U.S. Application Data
(60) Provisional application No. 61/264,431, filed on Nov. 25, 2009.

(51) Int. Cl. G01N 33/574; G01N 21/76; G01N 21/64; A61P 35/00; A61K 33/24; A61K 31/282; A61K 31/675; A61K 31/7048; C12Q 1/68; C40B 30/04

(52) U.S. Cl. 424/649; 435/7.1; 435/6.12; 435/6.11; 435/6.14; 435/7.92; 506/9; 514/492; 514/110; 514/27

(57) ABSTRACT

A cancer testis antigen biomarker useful to determine whether a non- small cell lung cancer tumor is likely to respond to neoadjuvant chemotherapy is provided. Methods of using the biomarker in the diagnosis, treatment and prognosis of non-small cell lung cancer also are provided.

### RELATED APPLICATION

This application claims the benefit under 35 USC §119(e) of U.S. provisional application Ser. No. 61/264,431, filed on Nov. 25, 2009, the entire contents of which are incorporated herein by reference.

### FIELD OF THE INVENTION

A cancer testis antigen biomarker useful to determine whether a non-small cell lung cancer tumor is likely to respond to chemotherapy, for example, neoadjuvant chemotherapy, is provided. Methods of using the biomarker in the diagnosis, treatment and prognosis of non-small cell lung cancer also are provided.

### BACKGROUND OF THE INVENTION

Lung cancer is the leading cause of cancer-related deaths worldwide. Lung cancer is often diagnosed at an advanced stage necessitating therapeutic surgery. Neoadjuvant chemotherapy is routinely administered to lung cancer patients prior to undergoing lobectomy or pneumonectomy. Whether or not a tumor reacts to chemotherapy, for example, neoadjuvant chemotherapy, has great impact on a lung cancer patient's survival time.

### SUMMARY OF THE INVENTION

The benefits of chemotherapy, for example, neoadjuvant chemotherapy, in non-small cell lung cancer (NSCLC) include early control of micrometastatic disease and an increased chance of tumor resectability. However, only a fraction of non-small cell lung cancer tumors respond favorably to chemotherapy. Accordingly, a significant proportion of patients receiving chemotherapy in NSCLC attain no clinical benefit. Further, chemotherapy, for example, neoadjuvant chemotherapy, is often associated with severe side effects that can result in a patient becoming unfit to undergo other therapeutic interventions, for example, surgery. At this time it is impossible to determine the likelihood of a NSCLC tumor to respond favorably to chemotherapy and, thus, it is impossible to diagnose a NSCLC patient as a candidate for chemotherapy, for example, neoadjuvant chemotherapy. Because of this lack of suitable diagnostic markers and methods, it is further impossible to administer chemotherapy selectively to those lung cancer patients diagnosed to be candidates for chemotherapy, while avoiding the risk of the associated side effects for those patients bearing a tumor determined to be unlikely to respond favorably to chemotherapy.

Some aspects of this invention provide a biomarker indicating tumor susceptibility to chemotherapy, for example, neoadjuvant chemotherapy, and methods of using such a biomarker in a clinical setting. Some aspects of this invention provide methods for determining and/or administering a suitable course of clinical intervention in NSCLC based on the analysis of a biomarker indicating a NSCLC tumor's susceptibility to chemotherapy.

The biomarkers and methods provided herein are useful in the diagnosis, staging, prognosis, selection and administration of treatment, of NSCLC.

Some aspects of this invention provide a method comprising determining a test level of NY-ESO-1 expression in a non-small cell lung cancer tumor of a subject, and comparing the test level of NY-ESO-1 expression to a control or reference level of NY-ESO-1 expression, wherein if the test level of NY-ESO-1 expression in the tumor is higher than the control or reference level of NY-ESO-1 expression, then the subject is indicated to be a candidate for chemotherapy, or wherein if the test level of NY-ESO-1 expression is similar or lower than the control or reference level of NY-ESO-1 expression, then the subject is indicated to not be a candidate for chemotherapy. Some aspects of this invention provide a method comprising obtaining a biopsy from a non-small cell lung cancer tumor of a subject, determining a test level of NY-ESO-1 expression in the biopsy, and comparing the test level of NY-ESO-1 expression to a control or reference level of NY-ESO-1 expression, wherein if the test level of NY-ESO-1 expression in the tumor is higher than the control or reference level of NY-ESO-1 expression, then the subject is indicated to be a candidate for chemotherapy, or wherein if the test level of NY-ESO-1 expression is similar or lower than the control or reference level of NY-ESO-1 expression, then the subject is indicated to not be a candidate for chemotherapy.

In some embodiments, the NY-ESO-1 expression is mRNA or protein expression. In some embodiments, the test level of NY-ESO-1 expression is determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay (ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS). In some embodiments, the subject has been diagnosed to have a stage II or stage III non-small cell lung cancer. In some embodiments, the chemotherapy is administered in temporal proximity to or in association with lobectomy, sleeve lobectomy or pneumonectomy.

In some embodiments, the method further comprises determining a second test level of NY-ESO-1 expression in a non-small cell lung cancer tumor of the subject after administration of the neoadjuvant chemotherapy. In some embodiments, if the second test level of NY-ESO-1 expression after administration of chemotherapy is lower than the test level of NY-ESO-1 expression before administration of chemotherapy, then the subject is indicated to have a median progression-free survival time expectancy, a progression-free survival time expectancy, and/or a projected progression-free survival time, of more than 1150 days and/or a median overall survival time expectancy, an overall survival time expectancy, and/or a projected overall survival time, of more than 1200 days. In some embodiments, if the second test level of NY-ESO-1 expression after administration of chemotherapy is similar or higher than the test level of NY-ESO-1 expression before administration of chemotherapy, then the subject is indicated to have a median progression-free survival time expectancy, a progression-free survival time expectancy, and/or a projected progression-free survival time, of less than 400 days and/or a median overall survival time expectancy, an overall survival time expectancy, and/or a projected overall survival time, of less than 750 days. In some embodiments, the chemotherapy is neoadjuvant chemotherapy.

Some aspects of this invention provide a method, comprising administering chemotherapy to a subject based on said subject being indicated or diagnosed to have a non-small cell lung cancer tumor expressing NY-ESO-1. Some aspects of this invention provide a method, comprising determining whether a non-small cell lung cancer tumor in a subject expresses NY-ESO-1, and administering chemotherapy to the subject based on the tumor expressing NY-ESO-1. Some aspects of this invention provide a method, comprising obtaining a biopsy from a non-small cell lung cancer tumor of a subject, determining a test level of NY-ESO-1 expression in the biopsy, and, if the biopsy expresses NY-ESO-1, administering chemotherapy to the subject, or, if the biopsy does not express NY-ESO-1, not administering chemotherapy to the subject. Some aspects of this invention provide a method, comprising determining whether a non-small cell lung cancer tumor in a subject expresses NY-ESO-1, and, if the tumor expresses NY-ESO-1, administering chemotherapy to the subject, or, if the tumor does not express NY-ESO-1, performing lobectomy or pneumonectomy without chemotherapy. Some aspects of this invention provide a method, comprising determining whether a non-small cell lung cancer tumor in a subject expresses NY-ESO-1, and, if the tumor expresses NY-ESO-1, administering chemotherapy to the subject, or, if the tumor does not express NY-ESO-1, administering healthcare other than chemotherapy. Some aspects of this invention provide a method, comprising determining a level of expression of NY-ESO-1 in a non-small cell lung cancer tumor of a subject, comparing said level to a control or reference level, and, if the level of expression in the tumor is higher than the control or reference level, administering chemotherapy, or, if the level of expression is similar or lower than the control or reference level, not administering chemotherapy.

In some embodiments, the NY-ESO-1 expression is mRNA or protein expression. In some embodiments, the test level of NY-ESO-1 expression is determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay (ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS). In some embodiments, the subject has been diagnosed to have a stage II or stage III non-small cell lung cancer. In some embodiments, the chemotherapy is administered in temporal proximity to or in association with lobectomy or pneumonectomy.

In some embodiments, the method further comprises determining a level of NY-ESO-1 expression in a non-small cell lung cancer tumor of the subject after administration of the chemotherapy. In some embodiments, if the level of expression after administration of chemotherapy is lower than the level of expression before administration of chemotherapy, then the subject is indicated to have a median progression-free survival time expectancy, a progression-free survival time expectancy, or a projected survival time, of more than 1150 days and/or a median overall survival time expectancy, an overall survival time expectancy, and/or a projected survival time, of more than 1200 days. In some embodiments, if the level of expression after administration of chemotherapy is similar or higher than the level of expression before administration of chemotherapy, then the subject is indicated to have a median progression-free survival time expectancy, a progression-free survival time expectancy, or a projected survival time, of less than 400 days and/or a median overall survival time expectancy, an overall survival time expectancy, and/or a projected survival time, of less than 750 days. In some embodiments, the chemotherapy is neoadjuvant chemotherapy.

Some aspects of this invention provide a method, comprising determining a level of expression of NY-ESO-1 in a non-small cell lung cancer tumor of a subject before and after administration of chemotherapy to the subject, comparing the level of expression in the tumor before administration of chemotherapy to the level of expression after administration of chemotherapy, and, if the level of expression after administration of chemotherapy is lower than the level of expression before administration of chemotherapy, then the subject is indicated to have a better-than-average median progression-free survival time expectancy, progression-free survival time expectancy, and/or projected progression-free survival time, and/or a better-than-average median overall survival time expectancy, overall survival time expectancy, and/or projected overall survival time, or, if the level of expression after administration of chemotherapy is similar or higher than the level of expression before administration of chemotherapy, then the subject is indicated to have a worse-than average median progression-free survival time expectancy, progression-free survival time expectancy, and/or projected progression-free survival time, and/or a worse-than-average median overall survival time expectancy, overall survival time expectancy, and/or projected overall survival time.

In some embodiments, if the level of expression after administration of chemotherapy is lower than the level of expression before administration of chemotherapy, then the subject is indicated to have a median progression-free survival time expectancy, progression-free survival time expectancy, and/or projected progression-free survival time, of more than 1150 days and/or a median overall survival time expectancy, overall survival time expectancy, and/or projected overall survival time of more than 1200 days. In some embodiments, if the level of expression after administration of chemotherapy is similar or higher than the level of expression before administration of chemotherapy, then the subject is indicated to have a median progression-free survival time expectancy, progression-free survival time expectancy, and/or projected progression-free survival time, of less than 400 days and/or a median overall survival time expectancy, overall survival time expectancy, and/or projected overall survival time, of less than 750 days. In some embodiments, the level of NY-ESO-1 expression is determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay

(ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS). In some embodiments, the subject has been diagnosed to have a stage II or stage III non-small cell lung cancer. In some embodiments, the chemotherapy is administered in temporal proximity to or in association with lobectomy or pneumonectomy. In some embodiments, the chemotherapy is neoadjuvant chemotherapy.

Some aspects of this invention provide a method of staging a non-small cell lung cancer tumor comprising determining whether the tumor expresses NY-ESO-1, and, if the tumor expresses NY-ESO-1, then the disease stage is determined to be “NY-ESO-1⁺” or “NY-ESO-1 positive”, or, if the tumor does not express NY-ESO-1, then the disease stage is determined to be “NY-ESO-1⁻” or “NY-ESO-1 negative”. In some embodiments, the method further comprises combining the disease stage determined based on NY-ESO-1 expression with other disease stage information for the same patient and administering health care based on the combined disease stage information. In some embodiments, the health care comprises chemotherapy if the tumor expresses NY-ESO-1. In some embodiments, the health care does not comprise chemotherapy if the tumor does not express NY-ESO-1. In some embodiments, the chemotherapy is neoadjuvant chemotherapy.

Some aspects of this invention provide a kit comprising an isolated NY-ESO-1 antibody, optionally labeled with a detection reagent, a reagent and/or buffer useful for storing and/or processing of a NSCLC biopsy sample, staining a NSCLC biopsy section or a

NSCLC tumor-derived cell population with the isolated NY-ESO-1 antibody, and/or instructions for the use of the isolated NY-ESO-1 antibody in determining the expression of NY-ESO-1 in a NSCLC biopsy or tumor-derived cell population.

These and other aspects and embodiments of the invention, as well as various advantages and utilities will be more apparent with respect to the drawings and detailed description of the invention.

### BRIEF DESCRIPTION OF THE DRAWINGS

FIG. 1. Association between pre-chemotherapy NY-ESO-1 expression and survival time.

FIG. 2. Association between NY-ESO-1 expression change and survival time.

### DETAILED DESCRIPTION

Cancer-Testis antigens (CTAgs) are a category of tumor antigens with expression restricted to male germ cells in the testis. CTAgs are expressed in a significant fraction of non-small cell lung cancer (NSCLC) tumors. Correlation of CTAg expression with the response of a tumor to chemotherapy, for example, neoadjuvant chemotherapy, in NSCLC has not previously been reported.

Some aspects of this invention relate to the identification of a CTAg of the group including NY-ESO-1, MAGE-A1, MAGE-A3, MAGE-C1, and MAGE-A4, as a biomarker in non-small cell lung cancer. Representative NCBI database entries relating to these CTAgs are NP_{—}001318.1 and NM_{—}001327.2 for NY-ESO-1; NM_{—}004988.4 and NP_{—}004979.3 for MAGE-A1; NM_{—}005362.3 and NP_{—}005353.1 for MAGE-A3; NM_{—}005462.4 and NP_{—}005453.2 for MAGE-C1; NM_{—}001011548.1 and NP_{—}001011548.1 for MAGE-A4; (www.ncbi.nlm.nih.gov). Some aspects of this invention relate to the discovery that expression of NY-ESO-1, MAGE-A1, MAGE-A3, MAGE-C1, and/or MAGE-A4, in a NSCLC tumor is a biomarker indicating tumor susceptibility to chemotherapy. Some aspects of this invention relate to the discovery that a NSCLC tumor that does not express NY-ESO-1, MAGE-A1, MAGE-A3, MAGE-C1, and/or MAGE-A4, is unlikely to show a desired clinical response to chemotherapy. Some aspects of this invention relate to the discovery that a NSCLC tumor that expresses NY-ESO-1, MAGE-A1, MAGE-A3, MAGE-C1, and/or MAGE-A4, at a low level or low levels is unlikely to show a desired clinical response to chemotherapy.

In some embodiments, expression of NY-ESO-1, MAGE-A1, MAGE-A3, MAGE-C1, and/or MAGE-A4, in a NSCLC tumor indicates that the tumor is likely to respond favorably to chemotherapy. In some embodiments, absence or a low level of expression of NY-ESO-1, MAGE-A1, MAGE-A3, MAGE-C1, and/or MAGE-A4 in a NSCLC tumor indicates that the tumor is unlikely to respond favorably to chemotherapy.

Some aspects of this invention relate to the identification of the CTAg NY-ESO-1 as a biomarker in non-small cell lung cancer. Some aspects of this invention relate to the discovery that NY-ESO-1 expression in a NSCLC tumor is a biomarker indicating tumor susceptibility to chemotherapy, for example, neoadjuvant chemotherapy. Some aspects of this invention relate to the discovery that a NSCLC tumor that does not express NY-ESO-1, or that expresses NY-ESO-1 at a low level, is unlikely to show a desired clinical response to chemotherapy, for example, neoadjuvant chemotherapy.

In some embodiments, NY-ESO-1 expression in a NSCLC tumor indicates that the tumor is likely to respond favorably to chemotherapy, for example, neoadjuvant chemotherapy. In some embodiments, absence of NY-ESO-1 expression or a low level of NY-ESO-1 expression in a NSCLC tumor indicates that the tumor is unlikely to respond favorably to chemotherapy, for example, neoadjuvant chemotherapy.

### Non-Small Cell Lung Cancer

Lung cancer is one of the most common cancers, accounting for approximately 15% of all cancer diagnoses and about 30% of all cancer deaths. It is the second most diagnosed cancer in men and women (after prostate and breast, respectively), and the leading cause of death from cancer each year in both men and women. Lung cancer can take many years to develop, and is, accordingly, mostly found in older people, with the average age at diagnosis being about 69 years.

Lung cancer is classified into two types, small cell lung cancer and non-small cell lung cancer, based on histological and cytological observations. About 80% of lung cancers are non-small cell lung cancers. Non-small cell lung cancer is further classified into subtypes, including, for example, squamous cell carcinoma, adenocarcinoma, bronchioalveolar carcinoma, and large cell undifferentiated carcinoma.

### NSCLC Staging

When lung cancer is diagnosed, a type, for example small cell lung cancer or a non-small cell lung cancer, and a disease stage is usually assigned. Disease stage is a form of classification that signifies the extent of the cancer. Disease staging is an important tool to determine the appropriate course of treatment, for example in determining whether or not a surgery is indicated and, if that is the case, what type of surgery (for example, lobectomy, sleeve lobectomy or pneumonectomy) is most likely to yield a desired result. A disease stage can either be a clinical stage or a pathological stage. Staging may be done using a variety of parameters and staging systems, for example, the tumor, node, and metastases (TNM) staging system, which takes into account the degree of spread of the primary tumor, the extent of regional lymph node involvement, and the presence or absence of distant metastases. According to the TNM staging system, there are four stages (I-IV) of non-small cell lung cancer.

In general, non-small cell lung cancer of stage I or II displays a size and location amenable for surgical removal. Stage I non-small cell lung cancer is characterized by a localized tumor, which has not spread to any lymph nodes. Stage II non-small cell lung cancer is characterized by a localized tumor, which has spread to a lymph node contained within the surrounding part of the lung. Stage III non-small cell lung cancer is characterized by a localized tumor, which has spread to a regional lymph node not contained within the lung, for example, a mediastinal lymph node. Stage III non-small cell lung cancer is further divided into two substages: stage IIIa, in which the lymph node metastasis is on the same side of the lung as the primary tumor, and stage IIIb, in which the cancer has spread to the opposite lung, to a lymph node above the collarbone, to the fluid surrounding the lungs, or in which the cancer grows into a vital structure of the chest. Stage IV non-small cell lung cancer is characterized by spreading of the cancer to different sections (lobes) of the lung, or to distant sites within the body, for example, to the brain, the bones, the liver, and/or in the adrenal glands.

Lung cancer is of often diagnosed at an advanced stage (II, III or IV) of the disease, because no screening methods able to detect early stages are available.

Some aspects of this invention relate to the addition of information regarding CTAg expression in a NSCLC tumor to the disease staging information for diagnostic and prognostic purposes. For example, in some embodiments a NSCLC tumor of stage IIIa, which is determined to express NY-ESO-1, is assigned stage IIIa NY-ESO⁺. As another example, in some embodiments, a NSCLC tumor of stage IIIa, which is determined to not express NY-ESO-1, is assigned stage IIIa NY-ESO-1⁻. In some embodiments, neoadjuvant chemotherapy is administered or not administered to a patient based on staging information including NY-ESO-1 expression status.

### “Downstaged” Versus “Not Downstaged”

The term “downstaged”, as used herein, refers to a tumor that is assessed to be in a lower disease stage after a clinical intervention, for example, after neoadjuvant chemotherapy, than the disease stage it was diagnosed to be in prior to the clinical intervention. For example, if a non-small cell lung cancer tumor that was diagnosed to be in stage III, for example according to the TNM staging system, is found to be in stage II after neoadjuvant chemotherapy, the tumor is referred to as a downstaged tumor.

The term “not downstaged”, accordingly, refers to a tumor that is assessed to be in the same or a higher disease stage after a clinical intervention than the disease stage it was diagnosed to be in prior to the clinical intervention. For example, if a non-small cell lung cancer tumor that was diagnosed to be in stage IIIa, for example according to the TNM staging system, is found to be in stage IIIc, IIIb, or IV after neoadjuvant chemotherapy, the tumor is referred to as a not downstaged tumor.

### NSCLC Treatment

Most stage I-II patients and many stage III NSCLC patients are treated with surgery to remove the tumor. More advanced tumor stages that are deemed non-resectable are usually treated with chemotherapy and/or radiotherapy.

In many non-small cell lung cancer patients, neoadjuvant chemotherapy (chemotherapy prior to surgery, sometimes also referred to as induction chemotherapy) is administered. One aim of neoadjuvant chemotherapy may be to reduce the size of the cancer before surgery, in order to render the procedure easier to perform and more likely to be successful. Another aim of neoadjuvant chemotherapy may be to turn a tumor from non-resectable to resectable status, for example, by shrinking the tumor volume. Another aim of neoadjuvant chemotherapy may be to generate a better distinction of tumor tissue and surrounding non-malignant or normal tissue to improve surgery results. Another aim of neoadjuvant chemotherapy is control of micrometastatic disease. A desired clinical response or a favorable response of a NSCLC to neoadjuvant chemotherapy may, therefore, be, for example, a reduction in tumor size, a better resolution between tumor and surrounding non-malignant or normal tissue, a change in tumor status from non-resectable to resectable, and/or a decrease in clinical stage (downstaging) of the tumor.

In some patients, the results of neoadjuvant chemotherapy may determine whether subsequent surgery is performed or not. For example, a patient diagnosed with a non-resectable tumor may only undergo surgery if the tumor responds favorably to neoadjuvant chemotherapy, rendering the tumor resectable. Unfortunately, a significant fraction of non-small cell lung cancer tumors does not respond favorably to neoadjuvant chemotherapy. Further, neoadjuvant chemotherapy can be highly toxic, and can be associated with severe side effects. In some cases reactions to neoadjuvant chemotherapy can be so severe that subsequent clinical interventions, for example surgery, are precluded because the patient is rendered unfit for anesthesia. While the benefits of neoadjuvant chemotherapy are very high for those patients carrying tumors that tumors respond favorably to it, there is also a high risk associated with neoadjuvant chemotherapy, namely rendering a patient unfit for subsequent surgery. This is particularly so in cases where a tumor initially diagnosed to be resectable does not respond to neoadjuvant chemotherapy.

The lack of a method to predict whether a particular NSCLC tumor is likely to respond to neoadjuvant chemotherapy, thus, constitutes an unmet clinical need.

Some aspects of this invention relate to a biomarker indicative of NSCLC tumor susceptibility and methods of using a biomarker to determine whether or not a NSCLC tumor patient is a candidate for neoadjuvant chemotherapy. In some embodiments, the biomarker is expression of NY-ESO-1 expression, in the tumor. In some embodiments, the biomarker is expression of NY-ESO-1 in the tumor at a higher level than a control or reference level. Biomarkers and diagnostic and therapeutic methods using biomarkers to determine susceptibility of a NSCLC tumor to neoadjuvant chemotherapy are described in more detail elsewhere herein.

### NSCLC Prognosis

The prognosis of non-small cell lung cancer disease course and outcome is affected by a number of factors, for example, the type and location of the cancer, the stage of the disease, the patient's general health, and the response to treatment. Since non-small cell lung cancer is often a terminal disease, an accurate prognosis, for example, of average life expectancy, would be of great benefit for many affected individuals. It is highly desirable, therefore, to identify and develop a biomarker allowing for a more precise determination of prognostic parameters, for example, projected overall survival time, progression free survival time, likelihood of tumor downstaging after neoadjuvant chemotherapy, likelihood of therapeutic success, etc.

Some aspects of this invention relate to prognostic methods using a biomarker to determine projected overall survival time, progression free survival time, likelihood of tumor downstaging after neoadjuvant chemotherapy, likelihood of therapeutic success, etc. in a NSCLC patient. In some embodiments, expression of NY-ESO-1 in a NSCLC tumor of a subject is used as a biomarker to prognose, for example, projected overall survival time, progression free survival time, likelihood of tumor downstaging after neoadjuvant chemotherapy, and/or likelihood of therapeutic success. In some embodiments, presence of NY-ESO-1 expression in a NSCLC tumor of a subject and/or NY-ESO-1 expression in a NSCLC tumor of a subject at a higher level than a control or reference level is indicative of the subject having an above-average projected overall survival time, progression free survival time, likelihood of tumor downstaging after neoadjuvant chemotherapy, and/or likelihood of therapeutic success. In some embodiments, absence of expression of NY-ESO-1 in a NSCLC tumor of a subject, failure to detect expression of NY-ESO-1 in a NSCLC tumor of a subject, and/or NY-ESO-1 expression in a NSCLC tumor of a subject at a lower level than a control or reference level and/or below the detection limit of a detection method employed, is indicative of the subject having a below-average projected overall survival time, progression free survival time, likelihood of tumor downstaging after neoadjuvant chemotherapy, and/or likelihood of therapeutic success.

In some embodiments, presence of NY-ESO-1 expression in a NSCLC tumor of a subject and/or NY-ESO-1 expression in a NSCLC tumor of a subject at a higher level than a control or reference level is indicative of the subject having a projected overall survival time and/or a projected progression free survival time of more than about 50 days, more than about 100 days, more than about 150 days, more than about 180 days, more than about 200 days, more than about 250 days, more than about 300 days, more than about 365 days, more than about 400 days, more than about 450 days, more than about 500 days, more than about 550 days, more than about 600 days, more than about 650 days, more than about 700 days, more than about 730 days, more than about 750 days, more than about 800 days, more than about 850 days, more than about 900 days, more than about 950 days, more than about 1000 days, more than about 1100 days, more than about 1200 days, more than about 1250 days, more than about 1300 days, more than about 1400 days, more than about 1460 days, or more than about 1500 days.

In some embodiments, absence of expression of NY-ESO-1 in a NSCLC tumor of a subject, failure to detect expression of NY-ESO-1 in a NSCLC tumor of a subject, and/or NY-ESO-1 expression in a NSCLC tumor of a subject at a lower level than a control or reference level and/or below the detection limit of a detection method employed, is indicative of the subject having a projected overall survival time and/or a projected progression free survival time of less than about 50 days, less than about 100 days, less than about 150 days, less than about 180 days, less than about 200 days, less than about 250 days, less than about 300 days, less than about 365 days, less than about 400 days, less than about 450 days, less than about 500 days, less than about 550 days, less than about 600 days, less than about 650 days, less than about 700 days, less than about 730 days, less than about 750 days, less than about 800 days, less than about 850 days, less than about 900 days, less than about 950 days, less than about 1000 days, less than about 1100 days, less than about 1200 days, less than about 1250 days, less than about 1300 days, less than about 1400 days, less than about 1460 days, or less than about 1500 days below the average overall survival time and/or the projected progression free survival time of NSCLC patients of the same disease stage.

In some embodiments, presence of NY-ESO-1 expression in a NSCLC tumor of a subject and/or NY-ESO-1 expression in a NSCLC tumor of a subject at a higher level than a control or reference level is indicative of the subject having a projected overall survival time and/or a projected progression free survival time of about 50 days, about 100 days, about 150 days, about 180 days, about 200 days, about 250 days, about 300 days, about 365 days, about 400 days, about 450 days, about 500 days, about 550 days, about 600 days, about 650 days, about 700 days, about 730 days, about 750 days, about 800 days, about 850 days, about 900 days, about 950 days, about 1000 days, about 1100 days, about 1200 days, about 1250 days, about 1300 days, about 1400 days, about 1460 days, or about 1500 days above the average overall survival time and/or the projected progression free survival time of NSCLC patients of the same disease stage. In some embodiments, presence of NY-ESO-1 expression in a NSCLC tumor of a subject and/or NY-ESO-1 expression in a NSCLC tumor of a subject at a higher level than a control or reference level is indicative of the subject having a projected overall survival time and/or a projected progression free survival time of more than 1500 days above the average overall survival time and/or the projected progression free survival time of NSCLC patients of the same disease stage.

In some embodiments, absence of expression of NY-ESO-1 in a NSCLC tumor of a subject, failure to detect expression of NY-ESO-1 in a NSCLC tumor of a subject, and/or NY-ESO-1 expression in a NSCLC tumor of a subject at a lower level than a control or reference level and/or below the detection limit of a detection method employed, is indicative of the subject having a projected overall survival time and/or a projected progression free survival time of about 50 days, about 100 days, about 150 days, about 180 days, about 200 days, about 250 days, about 300 days, about 365 days, about 400 days, about 450 days, about 500 days, about 550 days, about 600 days, about 650 days, about 700 days, about 730 days, about 750 days, about 800 days, about 850 days, about 900 days, about 950 days, about 1000 days, about 1100 days, about 1200 days, about 1250 days, about 1300 days, about 1400 days, about 1460 days, or about 1500 days below the average overall survival time and/or the projected progression free survival time of NSCLC patients of the same disease stage. In some embodiments, absence of expression of NY-ESO-1 in a NSCLC tumor of a subject, failure to detect expression of NY-ESO-1 in a NSCLC tumor of a subject, and/or NY-ESO-1 expression in a NSCLC tumor of a subject at a lower level than a control or reference level and/or below the detection limit of a detection method employed, is indicative of the subject having a projected overall survival time and/or a projected progression free survival time of more than 1500 days below the average overall survival time and/or the projected progression free survival time of NSCLC patients of the same disease stage.

### NY-ESO-1 as a Biomarker in NSCLC

Some aspects of this invention relate to the identification of NY-ESO-1 as a biomarker in non-small cell lung cancer. Some aspects of this invention relate to the use of the expression of NY-ESO-1 in a non-small cell lung cancer as a biomarker indicating whether or not the respective patient is a candidate for neoadjuvant chemotherapy. Some aspects of this invention relates to the use of the expression of NY-ESO-1 in non-small cell lung cancer is a biomarker indicating the likelihood of tumor downstaging after neoadjuvant chemotherapy. Some aspects of this invention relates to the use of the expression of NY-ESO-1 in non-small cell lung cancer as a biomarker indicating the range of prognostic factors for the respective patient, for example overall survival time expectancy and/or progression free survival time expectancy.

### NY-ESO-1

The term “NY-ESO-1” refers to cancer/testis antigen 1B (CTAG1B). Gene, transcript, and protein sequences of NY-ESO-1 are well known in the art. As a member of the cancer/testis antigen category, NY-ESO-1 is a tumor antigen that is not expressed in normal adult somatic tissue other than male germ cells in the testis. NY-ESO-1 sequences are well known in the art and can, for example, be found in public access databases (e.g., The National Center for Biotechnology Information (NCBI) at “www.ncbi.nlm.nih.gov”, The ENSEMBL Genome Browser at “www.ensembl.org”, or the UCSC Genome Browser at “genome.ucsc.edu”). A representative NY-ESO-1 protein sequence and a representative NY-ESO-1 transcript sequence from the NCBI database are given below:

>gi|4503119|ref|NP_001318.1|cancer/testis
antigen 1B [Homo sapiens]:
(SEQ ID NO: 1)
MQAEGRGTGGSTGDADGPGGPGIPDGPGGNAGGPGEAGATGGRGPRGAG
AARASGPGGGAPRGPHGGAASGLNGCCRCGARGPESRLLEFYLAMPFAT
PMEAELARRSLAQDAPPLPVPGVLLKEFTVSGNILTIRLTAADHRQLQL
SISSCLQQLSLLMWITQCFLPVFLAQPPSGQRR
>gi|215272337|ref|NM_001327.2| Homo sapiens cancer/
testis antigen 1B (CTAG1B), mRNA:
(SEQ ID NO: 2)
ATCCTCGTGGGCCCTGACCTTCTCTCTGAGAGCCGGGCAGAGGCTCCGG
AGCCATGCAGGCCGAAGGCCGGGGCACAGGGGGTTCGACGGGCGATGCT
GATGGCCCAGGAGGCCCTGGCATTCCTGATGGCCCAGGGGGCAATGCTG
GCGGCCCAGGAGAGGCGGGTGCCACGGGCGGCAGAGGTCCCCGGGGCGC
AGGGGCAGCAAGGGCCTCGGGGCCGGGAGGAGGCGCCCCGCGGGGTCCG
CATGGCGGCGCGGCTTCAGGGCTGAATGGATGCTGCAGATGCGGGGCCA
GGGGGCCGGAGAGCCGCCTGCTTGAGTTCTACCTCGCCATGCCTTTCGC
GACACCCATGGAAGCAGAGCTGGCCCGCAGGAGCCTGGCCCAGGATGCC
CCACCGCTTCCCGTGCCAGGGGTGCTTCTGAAGGAGTTCACTGTGTCCG
GCAACATACTGACTATCCGACTGACTGCTGCAGACCACCGCCAACTGCA
GCTCTCCATCAGCTCCTGTCTCCAGCAGCTTTCCCTGTTGATGTGGATC
ACGCAGTGCTTTCTGCCCGTGTTTTTGGCTCAGCCTCCCTCAGGGCAGA
GGCGCTAAGCCCAGCCTGGCGCCCCTTCCTAGGTCATGCCTCCTCCCCT
AGGGAATGGTCCCAGCACGAGTGGCCAGTTCATTGTGGGGGCCTGATTG
TTTGTCGCTGGAGGAGGACGGCTTACATGTTTGTTTCTGTAGAAAATAA
AACTGAGCTACGAAAAA

It will be appreciated by those of skill in the art, that, while the NY-ESO-1 sequences provided herein are representative, the scope of the invention is not limited to these sequences. The scope of methods provided by aspects of this invention extends, for example, to naturally occurring NY-ESO-1 variants, such as single nucleotide polymorphisms (SNPs), splice variants and other variants. NY-ESO-1 variants are well known to those in the art and can be identified in publicly available databases, for example, NY-ESO-1 SNPs can be identified in NCBI's SNP database at “www.ncbi.nlm.nih.gov/SNP”. Further, some detection methods useful in detecting and/or quantifying NY-ESO-1 expression in a cell or a tissue, do not distinguish between NY-ESO-1 variants. For example, an immunohistological method employing an NY-ESO-1-specific antibody to stain a cell or tissue will detect NY-ESO-1 variants as long as these variants are efficiently bound by the NY-ESO-1 antibody employed. Similarly, a nucleic acid hybridization-based assay for NY-ESO-1 expression, for example a northern blot, RT-PCR, or in situ hybridization assay, will detect any NY-ESO-1 variant that can efficiently hybridize to the NY-ESO-1 specific probe or primer employed. The term “NY-ESO-1 expression”, accordingly, is not limited to the expression of any of the sequences provided herein, but refers to the expression of a naturally occurring NY-ESO-1 gene or gene product. Further, it is well known to those of skill in the art, the detection of a NY-ESO-1 gene product (for example a NY-ESO-1 protein or transcript as exemplified herein) may comprise a determination of the gene product, or a fragment thereof. For example, RT-PCR based methods usually do not amplify whole transcript sequences but only fragments thereof that are sufficiently characteristic of the transcript to be detected to indicate expression of a specific transcript.

### Expression of NY-ESO-1 in NSCLC

In some embodiments, NY-ESO-1 expression in a NSCLC tumor is determined in a qualitative, semi-quantitative, or quantitative way. In some embodiments, NY-ESO-1 expression in a NSCLC tumor is determined by obtaining a cell or tissue sample of the tumor, for example from a tumor biopsy, and assaying the sample for NY-ESO-1 expression. In some embodiments, it is inferred that a NSCLC tumor expresses NY-ESO-1 if NY-ESO-1 expression is found in a sample obtained from the tumor.

A NSCLC tumor sample from a NSCLC patient may be obtained directly from the patient, for example by tumor biopsy, or from a third party, for example, a physician or hospital performing the biopsy procedure on the patient, handles, stores, archives, and/or processes the sample. Methods for performing NSCLC tumor biopsies are well known to those of skill in the art.

In some embodiments, NY-ESO-1 expression is determined in a qualitative way. For example, a NSCLC tumor may be determined to express NY-ESO-1, if an assay for an NY-ESO-1 gene product, for example a NY-ESO-1 protein or transcript, is positive. Similarly, a NSCLC tumor may be determined to not express NY-ESO-1, if an assay for an NY-ESO-1 gene product, for example a NY-ESO-1 protein or transcript, is negative. Qualitative assays for gene expression, for example assays for the detection of a specific protein or transcript, e.g. a NY-ESO-1 protein or transcript, are well known to those of skill in the art. Examples of assays for qualitative measurement of gene expression include, but are not limited to, immunohistochemistry, immunostaining, cytometry, FACS, ELISA, RT-PCR, microarray, northern blot, and western blot. In some embodiments, an assaying method that allows for quantitative or semi-quantitative measurement of gene expression, such as real-time PCR is used for qualitative measurement of gene expression. In some embodiments, a positive expression result (e.g. NY-ESO-1⁺) is determined if the readout of the respective assay is positive, if the readout is above the range of background usually observed in the employed assay, and/or if the readout is above the range of the readout expected or observed from a sample known to express the gene in question (e.g. NY-ESO-1). In some embodiments, a negative expression result (e.g. NY-ESO-1⁻) is determined if the readout of the respective assay is negative, if the readout is within the range of background usually observed in the employed assay, and/or if the readout is within the range of the readout expected or observed from a sample known to not express the gene in question (e.g. NY-ESO-1).

In some embodiments, NY-ESO-1 expression is determined in a quantitative or semi-quantitative way. In some embodiments, a level of NY-ESO-1 expression is determined. In some embodiments, the level of expression is an absolute level of expression. In some embodiments, the level of expression is a relative level of expression, for example a level of expression of NY-ESO-1 relative to a level of expression of another gene, for example a housekeeping gene (e.g. GAPDH, beta-Actin, etc.). In some embodiments the level of expression in a NSCLC tumor sample is measured relative to the level of expression of a plurality of genes in the same sample, to overall gene expression in the same sample, to overall protein content in the same sample or some other value useful for normalization. Methods to perform quantitative and/or semi-quantitative measurements of gene expression are well known to those of skill in the art and include, without limitation, immunohistochemistry, cytology, immunostaining, fluorescent activated cell sorting (FACS), ELISA, endpoint RT-PCR, semi-quantitative RT-PCR, real-time RT-PCR, western blot and northern blot.

In some embodiments, a cell population or a tissue from a tumor is obtained and stained with an antibody to detect NY-ESO-1 expression. In some embodiments, cells within a cell population or tissue are stained with an antibody to detect NY-ESO-1 expression and the fraction of NY-ESO-1 expressing cells is determined by a quantitative assay, for example, by counting stained cells under a microscope or by automated cell counting methods such as cytometry or FACS.

Details of suitable methods for determining gene expression in NSCLC tumors are well known to those of skill in the art. Exemplary methods are described, for example, in Driscoll, Lung Cancer: Volume 1: Molecular Pathology Methods and Reviews, and Lung Cancer: Volume 2: Diagnostic and Therapeutic Methods and Reviews, both by Humana Press, 2003, and included herein by reference.

### Diagnostics Using NY-ESO-1 Expression as a Biomarker

Some aspects of this invention relate to diagnostic methods employing NY-ESO-1 expression in a tumor as a biomarker to determine whether or not a non-small cell lung cancer patient is a candidate for neoadjuvant chemotherapy.

In some embodiments, a determination that a NSCLC tumor expresses NY-ESO-1 is indicative of the tumor to be likely to respond favorably to neoadjuvant chemotherapy, and/or the respective patient is indicated to be a candidate for neoadjuvant chemotherapy. In some embodiments, a determination that a NSCLC tumor does not express NY-ESO-1 is indicative of the tumor to be unlikely to respond favorably to neoadjuvant chemotherapy, and/or the respective patient is indicated to not be a candidate for neoadjuvant chemotherapy.

In some embodiments, an expression level, absolute or relative, of NY-ESO-1 in a NSCLC tumor is compared to a reference or control level. In some embodiments, if the expression level of NY-ESO-1 in a NSCLC tumor is higher than a reference or control level, then the tumor is indicated to be likely to respond favorably to neoadjuvant chemotherapy and/or the respective patient is indicated to be a candidate for neoadjuvant chemotherapy. In some embodiments, if the expression level of NY-ESO-1 in a NSCLC tumor is lower than a reference or control level, then the tumor is indicated to be unlikely to respond favorably to neoadjuvant chemotherapy and/or the respective patient is indicated to not be a candidate for neoadjuvant chemotherapy.

Determination of gene expression in a tumor can be effected via numerous methods known to those of skill in the art. In some embodiments, a tumor sample, for example, a tumor biopsy, may be obtained and the presence or absence and/or a level of expression may be determined by detecting an expression product of an NY-ESO-1 gene, for example a NY-ESO-1 protein or transcript. Examples of methods suitable for protein detection include, but are not limited to, immunohistology, cytology, cytometry, western blot, ELISA, cytology, FACS etc. Examples of methods suitable for transcript detection include, but are not limited to, northern blot, RT-PCR, in situ hybridization, expression profiling (e.g. microarray, massive parallel sequencing etc.). In some embodiments, NY-ESO-1 expression may be determined by a method involving staining cells or cell extracts with a binding agent that specifically binds a NY-ESO-1 protein. Suitable binding agents are well known in the art and examples of suitable binding agents include, but are not limited to, an antibody, an antibody fragment, an aptamer, or an adnectin. Suitable binding agents are well known in the art and include, for example, NY-ESO-1 antibodies and fragments as described in U.S. Pat. No. 6,252,052. Polyclonal and monoclonal antibodies specifically binding NY-ESO-1 are also commercially available, for example from Invitrogen (Cat #35-6200; and 18-2359), Santa Cruz Biotechnology (Cat #sc-53869(E978); sc-71734(6A146); and sc-71734(6A146)), Sigma-Aldrich (Cat #N2038), Spring Bioscience (Cat #E13714; and E13710), Thermo Scientific ((CAT #PA1-27404; PA1-38323; and PA1-38324), Lifespan Biosciences (Cat #LS-C17000; LS-C17002; LS-050403; and LS-C33082), Everest Biotech (Cat #EB09145), Abnova (Cat #PAB11785; and PAB 11786), and Anaspec (Cat #53726).

In some embodiments, a level of expression of NY-ESO-1 in a NSCLC tumor is determined. In some embodiments, the level of expression is measured as the percentage of cells in a tumor that express NY-ESO-1. For example, an immunohistological or cytological analysis may be performed using an antibody against NY-ESO-1, and the portion of cells staining positive for the gene product within a cell population may be determined. As another example, methods such as western blot, northern blot, RT-PCR, ELISA, FACS etc. can all yield quantifiable data. A level of NY-ESO-1 expression may, thus, be determined as the fraction of cells expressing NY-ESO-1 within a cell population, for example a NSCLC tumor cell population. Further, a level of NY-ESO-1 expression may be determined as the average expression level of NY-ESO-1 within a population of cells, for example in embodiments, in which a population of NSCLC tumor cells is obtained and assayed by methods without single cell resolution, e.g. RT-PCR, ELISA, western blot or northern blot.

A control or reference level, also referred to as baseline level can be determined using standard methods known to those of skill in the art. In some embodiments the control or reference level is a negative control or reference level, for example a level found or expected to be found in a cell or tissue of a non-NSCLC individual. Examples of methods for determining a control or reference level include, for example, determining a level of NY-ESO-1 in a cell or tissue from a non-NSCLC subject or determining an average or mean level of NY-ESO-1 in cells or tissues from a plurality of non-NSCLC subjects. Alternatively, a level of NY-ESO-1 may be determined in non-malignant tissue of a NSCLC patient, for example in non-malignant tissue surrounding a NSCLC tumor. In some embodiments, a control or reference level may be a historical value, a theoretical value, or an empirical value.

In some embodiments, the fraction of NY-ESO-1 expressing cells of a NSCLC tumor, for example as determined by immunohistochemistry, cytology, or cytometry, is about 5%, about 6-25%, about 10%, about 20%, about 25%, about 26-50%, about 30%, about 40%, about 50%, about 51-75%, about 60%, about 70%, about 75%, about 76-100%, about 80%, about 90%, or about 100% of a population of cells of the tumor, for example of a cell population of a NSCLC tumor examined by immunohistochemistry, cytology, or cytometry, indicating NY-ESO-1 expression in the tumor tissue being examined.

In some embodiments, the level of expression of NY-ESO-1 in a NSCLC tumor being investigated is at least about 5%, about 10%, 10-50%, about 20%, about 30%, about 40%, about 50%, 50-100%, about 60%, about 70%, about 80%, about 90%, about 100%, 100-150%, about 150%, 150-200%, about 2009o, 200-250%, about 250%, 250-500%, about 300%, about 400%, about 500%, 500-1000%, about 1000%, 1000-2500%, about 1500%, about 2000%, about 2500%, about 3000%, about 4000%, about 5000%, 5000%-10000%, about 6000%, about 7000%, about 8000%, about 9000%, or about 10000%, or more, greater than the level of NY-ESO-1 observed in negative control tissue or cell sample, indicating NY-ESO-1 expression in the tumor tissue being examined.

### Neoadjuvant Chemotherapy

In some embodiments, methods are provided for administration of chemotherapy, for example, of neoadjuvant chemotherapy, after a NSCLC tumor has been determined to express NY-ESO-1. In some embodiments, chemotherapy, for example, neoadjuvant chemotherapy, is administered to a subject having a NSCLC tumor, if the tumor expresses NY-ESO-1. In some embodiments, surgery is performed without neoadjuvant chemotherapy to a subject having a NSCLC tumor, if the tumor does not express NY-ESO-1. In some embodiments, chemotherapy, for example, neoadjuvant chemotherapy, is administered to a subject having a tumor that expresses NY-ESO-1 at a level higher than a reference or control level. In some embodiments, surgery without neoadjuvant chemotherapy is performed on a subject having a tumor that expresses NY-ESO-1 at a level lower or similar to a reference or control level. In some embodiments, chemotherapy, for example, neoadjuvant chemotherapy, is administered to a subject based on the subject having a NSCLC tumor expressing NY-ESO-1.

Chemotherapy is the administration of one or more anticancer drugs to destroy cancer cells and/or inhibit cancer cell proliferation. Chemotherapy can be administered alone or as an adjuvant therapy to improve the outcome of other clinical interventions, for example, of surgery or radiation therapy. In general, the term “adjuvant chemotherapy” refers to chemotherapy that is administered after a clinical intervention. Depending on the specific clinical scenario, adjuvant chemotherapy, is usually administered starting about four weeks after surgery. In contrast to adjuvant chemotherapy, “neoadjuvant chemotherapy” is chemotherapy that is given before a clinical intervention, for example, surgery or radiation therapy, to improve the outcome or, in some cases, enable the intended clinical intervention to be performed, for example, to render a non-resectable NSCLC tumor resectable. Specific schedules are followed during neoadjuvant chemotherapy, so that periods of treatment are accompanied by periods of recovery. Most neoadjuvant chemotherapy treatment schedules are completed within three to six months.

The parameters of chemotherapy, for example specific drug(s) used, schedule, number of cycles, dosage, dosage adjustment, timing, administration route, etc., depend, of course, on the specific scenario. Criteria for the determination of chemotherapeutic parameters are well known to those of skill in the art. For example, a patient's general health is a major factor determining whether or not a patient is a candidate for chemotherapy, for example, neoadjuvant chemotherapy. One measurement often employed to quantify a patients general health is performance status, for example as measured using the Karnofsky, ECOG/WHO/Zubrod, or Lansky scoring system. Further, a patient may be monitored during chemotherapy and changes to the initially proposed schedule may be made based on monitoring results. Methods for evaluating a NSCLC patient's health in regard to determining a chemotherapy schedule, for example by assessing a patient's general health and performance status, and for monitoring tumor and patient response to chemotherapy are well established in the art.

Chemotherapy may employ cytotoxic and/or cytostatic drugs (drugs that kill malignant cells, or inhibit their proliferation, respectively), including, for example, alkylating agents, antimetabolites, antitumor antibiotics, vinca alkaloids, taxanes, topoisomerase-I compounds, anthrapyrazoles, and epidophylotoxins. In addition, angiogenesis inhibiting drugs, including, for example, compounds that block growth promoting receptors (e.g., PDGF-R and VEGF-R) such as sunitinib (Sutent®) may be used. Non-limiting examples of drugs used for chemotherapy include: Cytoxan® (Cyclophosphamide), Methotrexate, 5-Fluorouracil (5-FU), Adriamycin® (Doxorubicin), Prednisone, Nolvadex® (Tamoxifen), Taxol® (Paclitaxel), Leucovorin, Oncovin® (Vincristine), Thioplex® (Thiotepa), Arimidex® (Anastrozole), Taxotere® (Docetaxel), Navelbine®, (Vinorelbine), Gemzar® (Gemcitabine), Ifex® (Ifosfamide), Pemetrexed, Topotecan, Melphalan (L-Pam®), Cisplatin (Cisplatinum®, Platinol®), Carboplatin (Paraplatin®), Carmustine (BCNU; BiCNU®), Methotrexate, Edatrexate, Mitomycin C (Mutamycin®), Mitoxantrone (Novantrone®), Vincristine (Oncovin®), Vinblastine (Velban®), Vinorelbine (Navelbine®), Fenretinide, Topotecan, Irinotecan, 9-amino-camptothecin [9-AC]; Biantrazole, Losoxantrone, Etoposide, and Teniposide.

Chemotherapy may comprise the administration of a single drug or the administration of a combination of drugs, for example, one of the following, commonly administered combinations: CMF (cyclophosphamide, methotrexate, and 5-fluorouracil); classic CMF (oral cyclophosphamide plus methotrexate and 5-fluorouracil); CAF or FAC (cyclophosphamide, Adriamycin® (doxorubicin), and 5-fluorouracil); AC (Adriamycin® and cyclophosphamide); ACT (Adriamycin® plus cyclophosphamide and tamoxifen); AC taxol (Adriamycin® plus cyclophosphamide and paclitaxel (Taxol®)); FACT (5-fluorouracil plus adriamycin®, cyclophosphamide, and tamoxifen); A-CMF or Adria/CMF (4 cycles of adriamycin® followed by 8 cycles of CMF); CMFP (CMF plus prednisone); CMFVP (CMF plus vincristine and prednisone); CAFMV (CAF plus methotrexate and vincristine); CMFVATN (CMF plus vincristine, adriamycin®, thiotepa, and tamoxifen); MF (methotrexate plus 5-fluorouracil and leucovorin).

Chemotherapeutic regimens and schedules, for example, for neoadjuvant chemotherapy in NSCLC patients, are well known in the art. An exemplary regimen useful in NSCLC neoadjuvant chemotherapy is described in Betticher et al., Journal of Clinical Oncology, 21(9), May 1, 2003, 1752-59, at page 1753, incorporated herein by reference.

Depending on the clinical scenario and the specific drug or combination of drugs employed, chemotherapy can be burdened with severe side effects, the most common of which include hair loss, mouth sores, loss of appetite, nausea and vomiting, low white blood cell counts, increased risk of infection, low blood platelet counts, increased risk of bruising or bleeding, low red blood cell counts, fatigue, neuropathy, heart damage, decrease in cognitive function, increased risk of leukemia, etc. In some cases, a side effect of chemotherapy may be severe enough to render a NSCLC patient unfit for surgery. The administration of chemotherapy, for example, neoadjuvant chemotherapy, thus, bears the risk of rendering a patient bearing a resectable tumor unfit for surgery. If a tumor does not respond favorably to the administered neoadjuvant chemotherapy and the respective patient is rendered unfit for surgery by a side effect of neoadjuvant chemotherapy, the time window for surgery may close before the patient recovers to a state considered fit for surgery, for example the tumor may grow from a resectable stage into a non-resectable stage. If a NSCLC tumor responds favorably to neoadjuvant chemotherapy and the respective patient is rendered unfit for surgery by a side effect of neoadjuvant chemotherapy, the desired clinical effect (e.g., reduction in tumor volume, improved distinction of malignant and non-malignant tissue, downstaging) may be lost by the time the patient recovers to a state fit for surgery.

Chemotherapy may necessitate the administration of additional medication to relieve side effects caused by chemotherapy including drugs that increase white blood cell counts, anti-anemia drugs (e.g., epoetin alfa (Procrit®, Epogen®)), cell-protecting drugs (e.g., amifostin (Ethyol®)), anti-nausea drugs, etc.

Neoadjuvant chemotherapy is generally administered to a NSCLC tumor patient as an adjuvant therapy to surgery. As a result, neoadjuvant chemotherapy is generally administered in association, or in temporal proximity prior to surgery. In some embodiments, there may be a time span of several weeks or months between administration of neoadjuvant chemotherapy and surgery, for example to give the respective patient time to recover from a side effect of the neoadjuvant chemotherapy. The surgery performed as therapeutic intervention in NSCLC is usually lobectomy or pneumonectomy.

Administration schedules, formulations, dosages and administration routes of drugs and compositions for neoadjuvant chemotherapy are well known to those in of skill in the art. Exemplary administration routes, schedules and dosages of commonly used chemotherapeutic drugs suitable for neoadjuvant chemotherapy drugs are described in chapter 33 (Chemotherapy of lung cancer), of Perry, The Chemotherapy Source Book, 4^{th} Edition, Lippinkott Williams & Wilkins, 2008, included herein by reference. The effects of neoadjuvant chemotherapy may be monitored using methods well established in the art, and/or methods provided herein.

While several embodiments of the present invention have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present invention. More generally, those skilled in the art will readily appreciate that all methods, reagents, and configurations described herein are meant to be exemplary and that the actual methods, reagents, and configurations will depend upon the specific application or applications for which the teachings of the present invention is/are used. Those skilled in the art will recognize, or be able to ascertain using no more than routine experimentation, many equivalents to the specific embodiments of the invention described herein. It is, therefore, to be understood that the embodiments described herein are presented by way of example only and that, within the scope of the appended claims and equivalents thereto, the invention may be practiced otherwise than as specifically described and claimed. The present invention is directed to each individual biomarker, gene, feature, system, article, material, reagent, kit, and/or method described herein. In addition, any combination of two or more such biomarkers, genes, features, systems, articles, materials, kits, and/or methods, if such features, systems, articles, materials, reagents, kits, and/or methods are not mutually inconsistent, is included within the scope of the present invention.

All references mentioned in the specifications are incorporated herein in their entirety by reference.

All definitions, as defined and used herein, should be understood to control over dictionary definitions, definitions in documents incorporated by reference, and/or ordinary meanings of the defined terms.

The indefinite articles “a” and “an”, as used herein in the specification and in the claims, unless clearly indicated to the contrary, should be understood to mean “at least one.”

The phrase “and/or,” as used herein in the specification and in the claims, should be understood to mean “ either or both” of the elements so conjoined, i.e., elements that are conjunctively present in some cases and disjunctively present in other cases. In cases where more than two elements are conjoined with the phrase “and/or”, it should be understood to mean “any element alone, any combination of two or more elements, or all elements” so conjoined. Other elements may optionally be present other than the elements specifically identified by the “and/or” clause, whether related or unrelated to those elements specifically identified unless clearly indicated to the contrary. Thus, as a non-limiting example, a reference to “A and/or B”, when used in conjunction with open-ended language such as “comprising” can refer, in one embodiment, to A without B (optionally including elements other than B); in another embodiment, to B without A (optionally including elements other than A); in yet another embodiment, to both A and B (optionally including other elements); etc.

As used herein in the specification and in the claims, “or” should be understood to have the same meaning as “and/or” as defined above. For example, when separating items in a list, “or” or “and/or” shall be interpreted as being inclusive, i.e., the inclusion of at least one, but also including more than one, of a number or list of elements, and, optionally, additional unlisted items. Only terms clearly indicated to the contrary, such as “only one of” or “exactly one of,” or, when used in the claims, “consisting of,” will refer to the inclusion of exactly one element of a number or list of elements. In general, the term “or” as used herein shall only be interpreted as indicating exclusive alternatives (i.e. “one or the other but not both”) when preceded by terms of exclusivity, such as “either,” “one of,” “only one of,” or “exactly one of.”

It should also be understood that, unless clearly indicated to the contrary, in any methods claimed herein that include more than one act, the order of the acts of the method is not necessarily limited to the order in which the acts of the method are recited.

### EXAMPLES

### Materials and Methods

### Experimental Design

Pre- and post-chemotherapy tumor samples for 24 consecutive patients receiving neoadjuvant chemotherapy for NSCLC were evaluated for five CTAgs (MAGE-A1, MAGE-A3, MAGE-A4, MAGE-C1, NY-ESO-1) by IHC.

Only patients with matched histology samples pre- and post-chemotherapy were included. Patients with cytological samples were excluded.

Immunohistology (IHC) expression levels were quantified as 0%, <5%, 6-25%, 26-50%, 51-75% and >75% (given as the proportion of positive cells/total cells in a tumor section). Change in CT antigen expression (at least a one step alteration in IHC expression levels) was correlated with chemotherapy response, which was classified as “downstaged” (characterized by a decrease in TNM stage grouping based on Sobin, TNM classification of malignant tumors, Wiley-Interscience, 7^{th} edition, 2009, incorporated herein by reference), or “not downstaged”.

Statistical analysis included estimation of progression-free survival (PFS) and overall survival (OS) by the Kaplan-Meier method using the log-rank test and Fisher's exact test to determine association between chemotherapy response and CT antigen expression.

### Protocol for CT Antigens

Monoclonal antibodies that specifically bind to NY-ESO-1 (E978), MAGE-A1 (MA545), MAGE-A3 (M3H67) were obtained from Ludwig Institute of Cancer Research and utilized at a dilutions 1:400, 1:50 and 1:20000 respectively. 57B, a culture supernatant monoclonal antibody for MAGE 4 was kindly supplied by Dr. G Spagnoli, Surgical Research Centre, Basel, Switzerland and used at a 1:100 dilution. A monoclonal antibody that specifically binds to MAGE-C1 (CT7-33) (DakoCytomation, Carpinteria, Calif.) was used at a 1:100 dilution.

Specimens of known positive tumors were used as a positive control and the relevant subclass control.

### Immunohistochemistry

Formalin-fixed paraffin sections of NSCLC tumor biopsies were prepared and dried overnight at 37° C. Following dewaxing in xylene and rehydration through alcohols, water bath retrieval was performed for 30 minutes using EDTA buffer pH 8.0 (NeoMarkers, Fremont, Calif.) for E978, MA545 and M3H67, Citrate buffer pH 6.0 (NeoMarkers, Fremont, Calif.) was used for CT7-33 and 57B.

Immunohistochemistry was performed using the Dako Envision+™ kit . All sections were submitted to 3% H₂O₂/PBS for 10 minutes to block endogenous peroxidase. All incubations were performed at room temperature using the Shandon Sequenza immunostainer. 3-amino-9-ethyl-carbazole (Sigma-Aldrich, St. Louis, Mo.) was used as the chromogen and slides were counterstained with Mayer's haematoxylin (Amber Scientific, Belmont, Wash.). Application of CrystalMount (Biomeda Corp., Calif.) preceded dehydration and mounting in DePeX (BDH 36125).

### Chemotherapy

A variety of chemotherapy regimens were used in patients in this study according to methods well known to those of skill in the art. Patients received 2-3 chemotherapeutic drugs at 3 weekly intervals and were typically given 2-3 cycles. A commonly used, exemplary regimen incorporated a platinum agent (for example, cisplatin or carboplatin) with a taxane (for example, docetaxel). For example, some patients received 40 mg/m²cisplatin on day 1 and 2, and 85 mg/m² docetaxel on day 1, every 3 weeks for three cycles (see, e.g., Betticher et al., Journal of Clinical Oncology, 21(9), May 1, 2003, 1752-1759). Another exemplary regimen included cyclophosphamide, etoposide and cisplatin.

### Patient Population

TABLE 1
Characterization of patient population.

| Variable | Outcome |
|---|---|
| Mean age | 65 |
| Gender | Male: 20 |
| | Female: 4 |
| Histology | Squamous: 8 |
| | Non-squamous: 16 |
| Pre-chemotherapy stage | Stage I: 0 |
| | Stage II: 3 |
| | Stage III: 21 |
| Post-chemotherapy stage | Stage I: 5 |
| | Stage II: 6 |
| | Stage III: 13 |
| Operation | Lobectomy: 17 |
| | Pneumonectomy: 7 |

### Results

### Pre-Chemotherapy CTAg Expression and Response to Neoadjuvant Chemotherapy

There was a statistically significant trend towards higher rates of downstaging in tumors that expressed NY-ESO-1 prior to neoadjuvant chemotherapy (44% vs. 0%; p=0.066). No tumors that failed to express NY-ESO-1 were downstaged following neoadjuvant chemotherapy. Statistical analysis revealed that there was no statistically significant association between pre-chemotherapy expression of other tested CTAgs and response to chemotherapy.

Further, there was a trend, albeit not statistically significant, towards higher rates of downstaging in tumors that expressed MAGE-A1 or MAGE-A4 (42% vs. 25%; and 40% vs. 29%, respectively).

TABLE 2
correlation of cancer testis antigen (CTAg
expression and downstaging after neoadjuvant chemotherapy.

| | | p-value (Fisher's |
|---|---|---|
| CTAg | Downstaged | exact test) |
| NY-ESO-1 | 44% (positive) | 0.066 |
| | vs. | |
| | 0% (negative) | |
| MAGE-A1 | 42% (positive) | NS |
| | vs. | |
| | 25% (negative) | |
| MAGE-A3 | 44% (positive) | NS |
| | vs. | |
| | 47% (negative) | |
| MAGE-C1 | 33% (positive) | NS |
| | vs. | |
| | 33% (negative) | |
| MAGE-A4 | 40% (positive) | NS |
| | vs. | |
| | 29% (negative) | |

### Post-Chemotherapy Change in CTAg Expression and Response to Neoadjuvant Chemotherapy

Tumors that had decreased NY-ESO-1 expression following chemotherapy had significantly higher rates of downstaging as compared to tumors that showed no decrease of NY-ESO-1 expression after chemotherapy (64% vs. 0%; p=0.002). There was a 64% downstaging rate in tumors exhibiting a decrease in NY-ESO-1 expression following chemotherapy and a 0% downstaging rate in tumors that had unchanged or increased NY-ESO-1 expression following chemotherapy. Statistical analysis revealed that there was no statistically significant association between change in expression levels of other CTAg and response to chemotherapy.

Further, tumors that had decreased MAGE-A1, MAGE-C1, or MAGE-A4 expression following chemotherapy had higher rates of downstaging, albeit not statistically significant, as compared to tumors that showed no decrease in expression of the respective CTAg (MAGE-A1: 40% vs. 32%; MAGE-C1: 50% vs. 32%; MAGE-A4: 43% vs. 29%).

TABLE 3
expression of CTAgs after neoadjuvant chemotherapy.

| | | p-value (Fisher's |
|---|---|---|
| CTAg | Expression | exact test) |
| NY-ESO-1 | Dec. expression: 64% | 0.002 |
| | vs. | |
| | Not dec. expression: 0% | |
| MAGE-A1 | Dec. expr.: 40% | NS |
| | vs. | |
| | Not dec. expr.: 32% | |
| MAGE-A3 | Dec. expr.: 20% | NS |
| | vs. | |
| | Not dec. expr.: 37% | |
| MAGE-C1 | Dec. expr.: 50%* | NS |
| | vs. | |
| | Not dec. expr.: 32% | |
| MAGE-A4 | Dec. expr.: 43% | NS |
| | vs. | |
| | Not dec. expr.: 29% | |

(Dec. = Decreased, exp. = expression).
*Only 2 patients had decreased MAGE-C1 expression post-chemotherapy

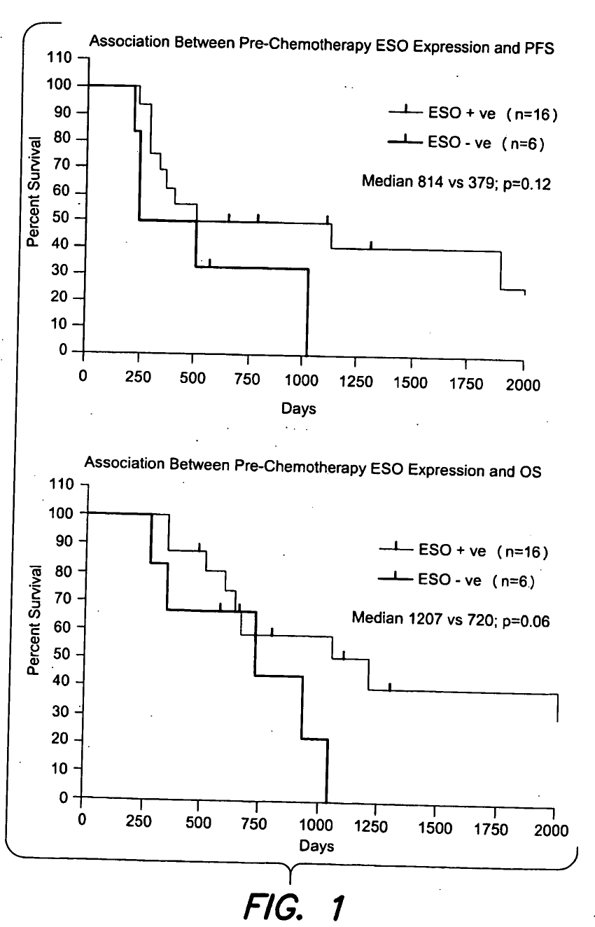

### Prognostic Impact of Pre-Chemotherapy NY-ESO-1 Expression (FIG. 1)

There was a non-significant trend towards longer progression-free survival (PS) (median 814 days vs. 379 days; p=0.12) and overall survival (OS) (median 1207 days vs. 720 days; p=0.06) in tumors that expressed NY-ESO-1 prior to chemotherapy.

There was no statistically significant difference between the two groups for age, gender, smoking status (current/ex vs. never), chemotherapy regimen (taxane vs. no taxane), operation (pneumonectomy vs. lobectomy), histology (squamous vs. non-squamous), tumor differentiation (poor vs. moderate/well), positive margins (yes or no), adjuvant chemotherapy (received vs. not received) or adjuvant radiotherapy (received vs. not received).

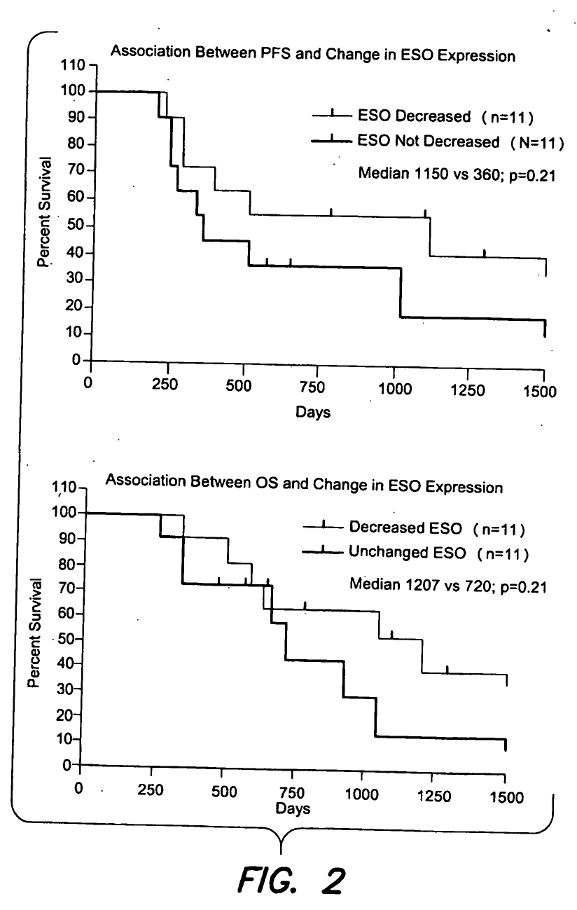

Prognostic Impact of Change in NY-ESO-1 Expression after Chemotherapy (FIG. 2)

There was a non-significant trend towards longer PS (median 1150 days vs. 360 days; p=0.21) and OS (median 1207 vs. 720 days; p=0.21) in tumors that had decreased NY-ESO-1 expression following chemotherapy.

There was no statistically significant difference between the two groups for age, gender, smoking status (current/ex vs. never) chemotherapy regimen (taxane vs. no taxane), operation (pneumonectomy vs. lobectomy), histology (squamous vs. non-squamous), tumor differentiation (poor vs. moderate/well), positive margins (yes or no), adjuvant chemotherapy (received vs. not received) or adjuvant radiotherapy (received vs. not received)

### Conclusions

Tumors that expressed NY-ESO-1 were more likely to be downstaged following neoadjuvant chemotherapy than tumors that did not express NY-ESO-1. Further, tumors that did not express NY-ESO-1 were unlikely to be downstaged following neoadjuvant chemotherapy. Tumors that had decreased NY-ESO-1 expression following chemotherapy were significantly more likely to be downstaged following neoadjuvant chemotherapy.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the scope of the invention. Accordingly, the foregoing description is by way of example only. All references described herein are incorporated by reference for the purposes described herein.

Moreover, this invention is not limited in its application to the details of construction and the arrangement of components set forth in the disclosed description. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

1. (canceled)

2. A method, comprising
obtaining a biopsy from a non-small cell lung cancer tumor of a subject,
determining a test level of NY-ESO-1 expression in the biopsy, wherein the test level of NY-ESO-1 expression is determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay (ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS), and
comparing the test level of NY-ESO-1 expression to a control or reference level of NY-ESO-1 expression,
wherein if the test level of NY-ESO-1 expression in the tumor is higher than the control or reference level of NY-ESO-1 expression, then the subject is indicated to be a candidate for chemotherapy, or
wherein if the test level of NY-ESO-1 expression is similar or lower than the control or reference level of NY-ESO-1 expression, then the subject is indicated to not be a candidate for chemotherapy.

3. The method of claim 1, wherein the NY-ESO-1 expression is mRNA or protein expression.

4. (canceled)

5. The method of claim 2, wherein the subject has been diagnosed to have a stage II or stage III non-small cell lung cancer.

6. The method of claim 2, wherein the chemotherapy is administered in temporal proximity to or in association with lobectomy, sleeve lobectomy or pneumonectomy.

7. The method of claim 2, further comprising determining a second test level of NY-ESO-1 expression in a non-small cell lung cancer tumor of the subject after administration of the chemotherapy.

8. (canceled)

9. The method of claim 7,
wherein if the second test level of NY-ESO-1 expression after administration of chemotherapy is lower than the test level of NY-ESO-1 expression before administration of chemotherapy, then the subject is indicated to have a progression-free survival time expectancy of more than 1150 days and/or an overall survival time expectancy of more than 1200 days, or
wherein if the second test level of NY-ESO-1 expression after administration of chemotherapy is similar or higher than the test level of NY-ESO-1 expression before administration of chemotherapy, then the subject is indicated to have a progression-free survival time expectancy of less than 400 days and/or a overall survival time expectancy of less than 750 days.

10. (canceled)

11. The method of claim 2, wherein the chemotherapy is neoadjuvant chemotherapy.

12. (canceled)

13. (canceled)

14. A method, comprising
obtaining a biopsy from a non-small cell lung cancer tumor of a subject,
determining a test level of NY-ESO-1 expression in the biopsy, wherein the test level of NY-ESO-1 expression is determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay (ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS), and
wherein if the biopsy expresses NY-ESO-1, administering chemotherapy to the subject, or
wherein if the biopsy does not express NY-ESO-1, not administering chemotherapy to the subject; or performing lobectomy or pneumonectomy without chemotherapy; or administering healthcare other than chemotherapy.

15. (canceled)

16. (canceled)

17. A method, comprising
determining a test level of expression of NY-ESO-1 in a non-small cell lung cancer tumor of a subject, wherein the test level of NY-ESO-1 expression is determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay (ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS), and
comparing said test level to a control or reference level,
wherein if the test level of expression in the tumor is higher than the control or reference level, administering chemotherapy, or
wherein if the test level of expression is similar or lower than the control or reference level, not administering chemotherapy.

18. The method of claim 14, wherein the NY-ESO-1 expression is mRNA or protein expression.

19. (canceled)

20. The method of claim 14, wherein the subject has been diagnosed to have a stage II or stage III non-small cell lung cancer.

21. The method of claim 14, wherein the chemotherapy is administered in temporal proximity to or in association with lobectomy or pneumonectomy.

22. The method of claim 14, further comprising determining a level of NY-ESO-1 expression in a non-small cell lung cancer tumor of the subject after administration of the chemotherapy.

23. (canceled)

24. (canceled)

25. The method of claim 22,
wherein if the level of expression after administration of neoadjuvant chemotherapy is lower than the level of expression before administration of neoadjuvant chemotherapy, then the subject is indicated to have a projected progression-free survival time of more than 1150 days and/or a projected overall survival time of more than 1200 days, or
wherein if the level of expression after administration of neoadjuvant chemotherapy is similar or higher than the level of expression before administration of neoadjuvant chemotherapy, then the subject is indicated to have a projected progression-free survival time of less than 400 days and/or a projected overall survival time of less than 750 days.

26. The method of claim 14, wherein the chemotherapy is neoadjuvant chemotherapy.

27. A method, comprising
determining a level of expression of NY-ESO-1 in a non-small cell lung cancer tumor of a subject before and after administration of chemotherapy to the subject, wherein the levels of NY-ESO-1 expression are determined by an immunohistological assay, a cytological assay, an mRNA expression assay, an RT-PCR assay, a northern blot assay, a protein expression assay, a western blotting assay, an enzyme-linked immunosorbent assay (ELISA), an enzyme-linked immunospot assay (ELISPOT), a lateral flow test assay, an enzyme immunoassay (EIA), a fluorescent polarization immunoassay (FPIA), a chemiluminescent immunoassay (CLIA), or a fluorescence activated cell sorting assay (FACS), and
comparing the level of expression in the tumor before administration of chemotherapy to the level of expression after administration of chemotherapy,
wherein if the level of expression after administration of chemotherapy is lower than the level of expression before administration of chemotherapy, then the subject is indicated to have a better-than-average median progression-free survival time expectancy and/or a better-than-average median overall survival time expectancy, or
wherein if the level of expression after administration of chemotherapy is similar or higher than the level of expression before administration of chemotherapy, then the subject is indicated to have a worse-than average median progression-free survival time expectancy and/or a worse-than-average median overall survival time expectancy.

28. The method of claim 27,
wherein if the level of expression after administration of chemotherapy is lower than the level of expression before administration of chemotherapy, then the subject is indicated to have a median progression-free survival time expectancy of more than 1150 days and/or a median overall survival time expectancy of more than 1200 days, or
wherein if the level of expression after administration of chemotherapy is similar or higher than the level of expression before administration of chemotherapy, then the subject is indicated to have a median progression-free survival time expectancy of less than 400 days and/or a median overall survival time expectancy of less than 750 days.

29. (canceled)

30. The method of claim 14, wherein the subject has been diagnosed to have a stage II or stage III non-small cell lung cancer.

31. The method of claim 14, wherein the chemotherapy is administered in temporal proximity to or in association with lobectomy or pneumonectomy.

32. The method of claim 14, wherein the chemotherapy is neoadjuvant chemotherapy.

33. -37. (canceled)


SEQUENCE LISTING (13 sequences)

SEQ ID NO: 1 - protein, 180 aa, Homo sapiens
  MISC_FEATURE (1)..(180): NY-ESO-1 protein (NP_001318.1)
MQAEGRGTGGSTGDADGPGGPGIPDGPGGNAGGPGEAGATGGRGPRGAGAARASGPGGGAPRGPHGGAASGLNGCCRCGARGPESRLLEFYLAMPFATPMEAELARRSLAQDAPPLPVPGVLLKEFTVSGNILTIRLTAADHRQLQLSISSCLQQLSLLMWITQCFLPVFLAQPPSGQRR

SEQ ID NO: 2 - DNA, 752 bp, Homo sapiens
  misc_feature (1)..(752): NY-ESO-1 cDNA (NM_001327.2)
atcctcgtgggccctgaccttctctctgagagccgggcagaggctccggagccatgcaggccgaaggccggggcacagggggttcgacgggcgatgctgatggcccaggaggccctggcattcctgatggcccagggggcaatgctggcggcccaggagaggcgggtgccacgggcggcagaggtccccggggcgcaggggcagcaagggcctcggggccgggaggaggcgccccgcggggtccgcatggcggcgcggcttcagggctgaatggatgctgcagatgcggggccagggggccggagagccgcctgcttgagttctacctcgccatgcctttcgcgacacccatggaagcagagctggcccgcaggagcctggcccaggatgccccaccgcttcccgtgccaggggtgcttctgaaggagttcactgtgtccggcaacatactgactatccgactgactgctgcagaccaccgccaactgcagctctccatcagctcctgtctccagcagctttccctgttgatgtggatcacgcagtgctttctgcccgtgtttttggctcagcctccctcagggcagaggcgctaagcccagcctggcgccccttcctaggtcatgcctcctcccctagggaatggtcccagcacgagtggccagttcattgtgggggcctgattgtttgtcgctggaggaggacggcttacatgtttgtttctgtagaaaataaaactgagctacgaaaaa

SEQ ID NO: 3 - DNA, 1755 bp, Homo sapiens
  misc_feature (1)..(1755): MAGE-A1 cDNA (NM_004988.4)
agagagaagcgaggtttccattctgagggacggcgtagagttcggccgaaggaacctgacccaggctctgtgaggaggcaaggttttcaggggacaggccaacccagaggacaggattccctggaggccacagaggagcaccaaggagaagatctgcctgtgggtcttcattgcccagctcctgcccacactcctgcctgctgccctgacgagagtcatcatgtctcttgagcagaggagtctgcactgcaagcctgaggaagcccttgaggcccaacaagaggccctgggcctggtgtgtgtgcaggctgccacctcctcctcctctcctctggtcctgggcaccctggaggaggtgcccactgctgggtcaacagatcctccccagagtcctcagggagcctccgcctttcccactaccatcaacttcactcgacagaggcaacccagtgagggttccagcagccgtgaagaggaggggccaagcacctcttgtatcctggagtccttgttccgagcagtaatcactaagaaggtggctgatttggttggttttctgctcctcaaatatcgagccagggagccagtcacaaaggcagaaatgctggagagtgtcatcaaaaattacaagcactgttttcctgagatcttcggcaaagcctctgagtccttgcagctggtctttggcattgacgtgaaggaagcagaccccaccggccactcctatgtccttgtcacctgcctaggtctctcctatgatggcctgctgggtgataatcagatcatgcccaagacaggcttcctgataattgtcctggtcatgattgcaatggagggcggccatgctcctgaggaggaaatctgggaggagctgagtgtgatggaggtgtatgatgggagggagcacagtgcctatggggagcccaggaagctgctcacccaagatttggtgcaggaaaagtacctggagtaccggcaggtgccggacagtgatcccgcacgctatgagttcctgtggggtccaagggccctcgctgaaaccagctatgtgaaagtccttgagtatgtgatcaaggtcagtgcaagagttcgctttttcttcccatccctgcgtgaagcagctttgagagaggaggaagagggagtctgagcatgagttgcagccaaggccagtgggagggggactgggccagtgcaccttccagggccgcgtccagcagcttcccctgcctcgtgtgacatgaggcccattcttcactctgaagagagcggtcagtgttctcagtagtaggtttctgttctattgggtgacttggagatttatctttgttctcttttggaattgttcaaatgtttttttttaagggatggttgaatgaacttcagcatccaagtttatgaatgacagcagtcacacagttctgtgtatatagtttaagggtaagagtcttgtgttttattcagattgggaaatccattctattttgtgaattgggataataacagcagtggaataagtacttagaaatgtgaaaaatgagcagtaaaatagatgagataaagaactaaagaaattaagagatagtcaattcttgccttatacctcagtctattctgtaaaatttttaaagatatatgcatacctggatttccttggcttctttgagaatgtaagagaaattaaatctgaataaagaattcttcctgttaaaaaaaaaaaaaaaaaaaaaaaaaaaaaaaaaaaaaaaaaaaaaa

SEQ ID NO: 4 - protein, 309 aa, Homo sapiens
  MISC_FEATURE (1)..(309): MAGE-A1 protein (NP_004979.3)
MSLEQRSLHCKPEEALEAQQEALGLVCVQAATSSSSPLVLGTLEEVPTAGSTDPPQSPQGASAFPTTINFTRQRQPSEGSSSREEEGPSTSCILESLFRAVITKKVADLVGFLLLKYRAREPVTKAEMLESVIKNYKHCFPEIFGKASESLQLVFGIDVKEADPTGHSYVLVTCLGLSYDGLLGDNQIMPKTGFLIIVLVMIAMEGGHAPEEEIWEELSVMEVYDGREHSAYGEPRKLLTQDLVQEKYLEYRQVPDSDPARYEFLWGPRALAETSYVKVLEYVIKVSARVRFFFPSLREAALREEEEGV

SEQ ID NO: 5 - DNA, 1753 bp, Homo sapiens
  misc_feature (1)..(1753): MAGE-A3 cDNA (NM_005362.3)
gagattctcgccctgagcaacgagcgacggcctgacgtcggcggagggaagccggcccaggctcggtgaggaggcaaggttctgaggggacaggctgacctggaggaccagaggcccccggaggagcactgaaggagaagatctgccagtgggtctccattgcccagctcctgcccacactcccgcctgttgccctgaccagagtcatcatgcctcttgagcagaggagtcagcactgcaagcctgaagaaggccttgaggcccgaggagaggccctgggcctggtgggtgcgcaggctcctgctactgaggagcaggaggctgcctcctcctcttctactctagttgaagtcaccctgggggaggtgcctgctgccgagtcaccagatcctccccagagtcctcagggagcctccagcctccccactaccatgaactaccctctctggagccaatcctatgaggactccagcaaccaagaagaggaggggccaagcaccttccctgacctggagtccgagttccaagcagcactcagtaggaaggtggccgagttggttcattttctgctcctcaagtatcgagccagggagccggtcacaaaggcagaaatgctggggagtgtcgtcggaaattggcagtatttctttcctgtgatcttcagcaaagcttccagttccttgcagctggtctttggcatcgagctgatggaagtggaccccatcggccacttgtacatctttgccacctgcctgggcctctcctacgatggcctgctgggtgacaatcagatcatgcccaaggcaggcctcctgataatcgtcctggccataatcgcaagagagggcgactgtgcccctgaggagaaaatctgggaggagctgagtgtgttagaggtgtttgaggggagggaagacagtatcttgggggatcccaagaagctgctcacccaacatttcgtgcaggaaaactacctggagtaccggcaggtccccggcagtgatcctgcatgttatgaattcctgtggggtccaagggccctcgttgaaaccagctatgtgaaagtcctgcaccatatggtaaagatcagtggaggacctcacatttcctacccacccctgcatgagtgggttttgagagagggggaagagtgagtctgagcacgagttgcagccagggccagtgggagggggtctgggccagtgcaccttccggggccgcatcccttagtttccactgcctcctgtgacgtgaggcccattcttcactctttgaagcgagcagtcagcattcttagtagtgggtttctgttctgttggatgactttgagattattctttgtttcctgttggagttgttcaaatgttccttttaacggatggttgaatgagcgtcagcatccaggtttatgaatgacagtagtcacacatagtgctgtttatatagtttaggagtaagagtcttgttttttactcaaattgggaaatccattccattttgtgaattgtgacataataatagcagtggtaaaagtatttgcttaaaattgtgagcgaattagcaataacatacatgagataactcaagaaatcaaaagatagttgattcttgccttgtacctcaatctattctgtaaaattaaacaaatatgcaaaccaggatttccttgacttctttgagaatgcaagcgaaattaaatctgaataaataattcttcctcttcaaaaaaaaaaaaaaaaaaaaaaaaaaaaaa

SEQ ID NO: 6 - protein, 314 aa, Homo sapiens
  MISC_FEATURE (1)..(314): MAGE-A3 protein (NP_005353.1)
MPLEQRSQHCKPEEGLEARGEALGLVGAQAPATEEQEAASSSSTLVEVTLGEVPAAESPDPPQSPQGASSLPTTMNYPLWSQSYEDSSNQEEEGPSTFPDLESEFQAALSRKVAELVHFLLLKYRAREPVTKAEMLGSVVGNWQYFFPVIFSKASSSLQLVFGIELMEVDPIGHLYIFATCLGLSYDGLLGDNQIMPKAGLLIIVLAIIAREGDCAPEEKIWEELSVLEVFEGREDSILGDPKKLLTQHFVQENYLEYRQVPGSDPACYEFLWGPRALVETSYVKVLHHMVKISGGPHISYPPLHEWVLREGEE

SEQ ID NO: 7 - DNA, 4337 bp, Homo sapiens
  misc_feature (1)..(4337): MAGE-C1 cDNA (NM_005462.4)
gctttgccggatgtgctttcccggcggccatcttgggagtctgaaggacctgaggcattttgtgacgaggatcgtctcaggtcagcggagggaggagacttatagacctatccagtcttcaaggtgctccagaaagcaggagttgaagacctgggtgtgagggacacatacatcctaaaagcaccacagcagaggaggcccaggcagtgccaggagtcaaggttcccagaagacaaaccccctaggaagacaggcgacctgtgaggccctagagcaccaccttaagagaagaagagctgtaagccggcctttgtcagagccatcatgggggacaaggatatgcctactgctgggatgccgagtcttctccagagttcctctgagagtcctcagagttgtcctgagggggaggactcccagtctcctctccagattccccagagttctcctgagagcgacgacaccctgtatcctctccagagtcctcagagtcgttctgagggggaggactcctcggatcctctccagagacctcctgaggggaaggactcccagtctcctctccagattccccagagttctcctgagggcgacgacacccagtctcctctccagaattctcagagttctcctgaggggaaggactccctgtctcctctagagatttctcagagccctcctgagggtgaggatgtccagtctcctctgcagaatcctgcgagttccttcttctcctctgctttattgagtattttccagagttcccctgagagtactcaaagtccttttgagggttttccccagtctgttctccagattcctgtgagcgccgcctcctcctccactttagtgagtattttccagagttcccctgagagtactcaaagtccttttgagggttttccccagtctccactccagattcctgtgagccgctccttctcctccactttattgagtattttccagagttcccctgagagaactcagagtacttttgagggttttgcccagtctcctctccagattcctgtgagcccctcctcctcctccactttactgagtcttttccagagtttctctgagagaactcagagtacttttgagggttttgcccagtcttctctccagattcctgtgagcccctccttctcctccactttagtgagtcttttccagagttcccctgagagaactcagagtacttttgagggttttccccagtctcctctccagattcctgtgagctcctcctcctcctccactttattgagtcttttccagagttcccctgagagaactcacagtacttttgagggttttccccagtctcttctccagattcctatgacctcctccttctcctctactttattgagtattttccagagttctcctgagagtgctcaaagtacttttgagggttttccccagtctcctctccagattcctgggagcccctccttctcctccactttactgagtcttttccagagttcccctgagagaactcacagtacttttgagggttttccccagtctcctctccagattcctatgacctcctccttctcctctactttattgagtattttacagagttctcctgagagtgctcaaagtgcttttgagggttttccccagtctcctctccagattcctgtgagctcctctttctcctacactttattgagtcttttccagagttcccctgagagaactcacagtacttttgagggttttccccagtctcctctccagattcctgtgagctcctcctcctcctcctccactttattgagtcttttccagagttcccctgagtgtactcaaagtacttttgagggttttccccagtctcctctccagattcctcagagtcctcctgaaggggagaatacccattctcctctccagattgttccaagtcttcctgagtgggaggactccctgtctcctcactactttcctcagagccctcctcagggggaggactccctatctcctcactactttcctcagagccctcctcagggggaggactccctgtctcctcactactttcctcagagccctcagggggaggactccctgtctcctcactactttcctcagagccctcctcagggggaggactccatgtctcctctctactttcctcagagtcctcttcagggggaggaattccagtcttctctccagagccctgtgagcatctgctcctcctccactccatccagtcttccccagagtttccctgagagttctcagagtcctcctgaggggcctgtccagtctcctctccatagtcctcagagccctcctgaggggatgcactcccaatctcctctccagagtcctgagagtgctcctgagggggaggattccctgtctcctctccaaattcctcagagtcctcttgagggagaggactccctgtcttctctccattttcctcagagtcctcctgagtgggaggactccctctctcctctccactttcctcagtttcctcctcagggggaggacttccagtcttctctccagagtcctgtgagtatctgctcctcctccacttctttgagtcttccccagagtttccctgagagtcctcagagtcctcctgaggggcctgctcagtctcctctccagagacctgtcagctccttcttctcctacactttagcgagtcttctccaaagttcccatgagagtcctcagagtcctcctgaggggcctgcccagtctcctctccagagtcctgtgagctccttcccctcctccacttcatcgagtctttcccagagttctcctgtgagctccttcccctcctccacttcatcgagtctttccaagagttcccctgagagtcctctccagagtcctgtgatctccttctcctcctccacttcattgagcccattcagtgaagagtccagcagcccagtagatgaatatacaagttcctcagacaccttgctagagagtgattccttgacagacagcgagtccttgatagagagcgagcccttgttcacttatacactggatgaaaaggtggacgagttggcgcggtttcttctcctcaaatatcaagtgaagcagcctatcacaaaggcagagatgctgacgaatgtcatcagcaggtacacgggctactttcctgtgatcttcaggaaagcccgtgagttcatagagatactttttggcatttccctgagagaagtggaccctgatgactcctatgtctttgtaaacacattagacctcacctctgaggggtgtctgagtgatgagcagggcatgtcccagaaccgcctcctgattcttattctgagtatcatcttcataaagggcacctatgcctctgaggaggtcatctgggatgtgctgagtggaataggggtgcgtgctgggagggagcactttgcctttggggagcccagggagctcctcactaaagtttgggtgcaggaacattacctagagtaccgggaggtgcccaactcttctcctcctcgttacgaattcctgtggggtccaagagctcattcagaagtcattaagaggaaagtagtagagtttttggccatgctaaagaataccgtccctattacctttccatcctcttacaaggatgctttgaaagatgtggaagagagagcccaggccataattgacaccacagatgattcgactgccacagaaagtgcaagctccagtgtcatgtcccccagcttctcttctgagtgaagtctagggcagattcttccctctgagtttgaagggggcagtcgagtttctacgtggtggagggcctggttgaggctggagagaacacagtgctatttgcatttctgttccatatgggtagttatggggtttacctgttttacttttgggtatttttcaaatgcttttcctattaataacaggtttaaatagcttcagaatcctagtttatgcacatgagtcgcacatgtattgctgtttttctggtttaagagtaacagtttgatattttgtaaaaacaaaaacacacccaaacacaccacattgggaaaaccttctgcctcattttgtgatgtgtcacaggttaatgtggtgttactgtaggaattttcttgaaactgtgaaggaactctgcagttaaatagtggaataaagtaaaggattgttaatgtttgcatttcctcaggtcctttagtctgttgttcttgaaaactaaagatacatacctggtttgcttggcttacgtaagaaagtagaagaaagtaaactgtaataaataaaagtgtcagtgactcatttatttgatgaaaaaaaaaaaaaaaaaa

SEQ ID NO: 8 - protein, 1142 aa, Homo sapiens
  MISC_FEATURE (1)..(1142): MAGE-C1 protein (NP_005453.2)
MGDKDMPTAGMPSLLQSSSESPQSCPEGEDSQSPLQIPQSSPESDDTLYPLQSPQSRSEGEDSSDPLQRPPEGKDSQSPLQIPQSSPEGDDTQSPLQNSQSSPEGKDSLSPLEISQSPPEGEDVQSPLQNPASSFFSSALLSIFQSSPESTQSPFEGFPQSVLQIPVSAASSSTLVSIFQSSPESTQSPFEGFPQSPLQIPVSRSFSSTLLSIFQSSPERTQSTFEGFAQSPLQIPVSPSSSSTLLSLFQSFSERTQSTFEGFAQSSLQIPVSPSFSSTLVSLFQSSPERTQSTFEGFPQSPLQIPVSSSSSSTLLSLFQSSPERTHSTFEGFPQSLLQIPMTSSFSSTLLSIFQSSPESAQSTFEGFPQSPLQIPGSPSFSSTLLSLFQSSPERTHSTFEGFPQSPLQIPMTSSFSSTLLSILQSSPESAQSAFEGFPQSPLQIPVSSSFSYTLLSLFQSSPERTHSTFEGFPQSPLQIPVSSSSSSSTLLSLFQSSPECTQSTFEGFPQSPLQIPQSPPEGENTHSPLQIVPSLPEWEDSLSPHYFPQSPPQGEDSLSPHYFPQSPPQGEDSLSPHYFPQSPQGEDSLSPHYFPQSPPQGEDSMSPLYFPQSPLQGEEFQSSLQSPVSICSSSTPSSLPQSFPESSQSPPEGPVQSPLHSPQSPPEGMHSQSPLQSPESAPEGEDSLSPLQIPQSPLEGEDSLSSLHFPQSPPEWEDSLSPLHFPQFPPQGEDFQSSLQSPVSICSSSTSLSLPQSFPESPQSPPEGPAQSPLQRPVSSFFSYTLASLLQSSHESPQSPPEGPAQSPLQSPVSSFPSSTSSSLSQSSPVSSFPSSTSSSLSKSSPESPLQSPVISFSSSTSLSPFSEESSSPVDEYTSSSDTLLESDSLTDSESLIESEPLFTYTLDEKVDELARFLLLKYQVKQPITKAEMLTNVISRYTGYFPVIFRKAREFIEILFGISLREVDPDDSYVFVNTLDLTSEGCLSDEQGMSQNRLLILILSIIFIKGTYASEEVIWDVLSGIGVRAGREHFAFGEPRELLTKVWVQEHYLEYREVPNSSPPRYEFLWGPRAHSEVIKRKVVEFLAMLKNTVPITFPSSYKDALKDVEERAQAIIDTTDDSTATESASSSVMSPSFSSE

SEQ ID NO: 9 - DNA, 1724 bp, Homo sapiens
  misc_feature (1)..(1724): MAGE-A1 cDNA (NM_001011548.1; transcript variant 1)
agagacaagcgagcttctgcgtctgactcgcagcttgagactggcggagggaagcccgcccaggctctataaggagacaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 10 - DNA, 1727 bp, Homo sapiens
  misc_feature: MAGE-A4 cDNA (NM_002362.4; transcript variant 2)
ggttagagagaagcgagctgctctgtctgaccagcagcttgggattggcggagggaagcgggccaggccctgtgaggagtcaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 11 - DNA, 1721 bp, Homo sapiens
  misc_feature: MAGE-A4 cDNA (NM_001011549.1; transcript variant 3)
ttagagagaagcgagctgctctgaccagccgcttgggattggcggagggaagcgggccaggccctgtgaggagtcaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 12 - DNA, 1712 bp, Homo sapiens
  misc_feature: MAGE-A4 cDNA (NM_001011550.1; transcript variant 4)
cgagctgctgtctgaccagcagcttgggattggtggaaggaagcaggccaggccctgtgaggagtcaaggttctgagcagacaggccaaccggaggacaggattccctggaggccacagaggagcaccaaggagaagatctgcctgtgggtccccattgcccagcttttgcctgcactcttgcctgctgccctgaccagagtcatcatgtcttctgagcagaagagtcagcactgcaagcctgaggaaggcgttgaggcccaagaagaggccctgggcctggtgggtgcacaggctcctactactgaggagcaggaggctgctgtctcctcctcctctcctctggtccctggcaccctggaggaagtgcctgctgctgagtcagcaggtcctccccagagtcctcagggagcctctgccttacccactaccatcagcttcacttgctggaggcaacccaatgagggttccagcagccaagaagaggaggggccaagcacctcgcctgacgcagagtccttgttccgagaagcactcagtaacaaggtggatgagttggctcattttctgctccgcaagtatcgagccaaggagctggtcacaaaggcagaaatgctggagagagtcatcaaaaattacaagcgctgctttcctgtgatcttcggcaaagcctccgagtccctgaagatgatctttggcattgacgtgaaggaagtggaccccgccagcaacacctacacccttgtcacctgcctgggcctttcctatgatggcctgctgggtaataatcagatctttcccaagacaggccttctgataatcgtcctgggcacaattgcaatggagggcgacagcgcctctgaggaggaaatctgggaggagctgggtgtgatgggggtgtatgatgggagggagcacactgtctatggggagcccaggaaactgctcacccaagattgggtgcaggaaaactacctggagtaccggcaggtacccggcagtaatcctgcgcgctatgagttcctgtggggtccaagggctctggctgaaaccagctatgtgaaagtcctggagcatgtggtcagggtcaatgcaagagttcgcattgcctacccatccctgcgtgaagcagctttgttagaggaggaagagggagtctgagcatgagttgcagccagggctgtggggaaggggcagggctgggccagtgcatctaacagccctgtgcagcagcttcccttgcctcgtgtaacatgaggcccattcttcactctgtttgaagaaaatagtcagtgttcttagtagtgggtttctattttgttggatgacttggagatttatctctgtttccttttacaattgttgaaatgttccttttaatggatggttgaattaacttcagcatccaagtttatgaatcgtagttaacgtatattgctgttaatatagtttaggagtaagagtcttgttttttattcagattgggaaatccgttctattttgtgaatttgggacataataacagcagtggagtaagtatttagaagtgtgaattcaccgtgaaataggtgagataaattaaaagatacttaattcccgccttatgcctcagtctattctgtaaaatttaaaaaatatatatgcatacctggatttccttggcttcgtgaatgtaagagaaattaaatctgaataaataattctttctgttaa

SEQ ID NO: 13 - protein, 317 aa, Homo sapiens
  MISC_FEATURE (1)..(317): MAGE-A4 (NP_001011548.1; NP_002353.3; NP_001011549.1; NP_001011550.1)
MSSEQKSQHCKPEEGVEAQEEALGLVGAQAPTTEEQEAAVSSSSPLVPGTLEEVPAAESAGPPQSPQGASALPTTISFTCWRQPNEGSSSQEEEGPSTSPDAESLFREALSNKVDELAHFLLRKYRAKELVTKAEMLERVIKNYKRCFPVIFGKASESLKMIFGIDVKEVDPASNTYTLVTCLGLSYDGLLGNNQIFPKTGLLIIVLGTIAMEGDSASEEEIWEELGVMGVYDGREHTVYGEPRKLLTQDWVQENYLEYRQVPGSNPARYEFLWGPRALAETSYVKVLEHVVRVNARVRIAYPSLREAALLEEEEGV